(19) United States
(12) Patent Application Publication
(10) Pub. No.: US 2011/0052507 A1
(43) Pub. Date: Mar. 3, 2011

(54) ORAL CARE COMPOSITIONS COMPRISING CASEIN, OVALBUMIN, WHEY OR SOY PROTEIN

(76) Inventors: Michele Emily Barbour, Avon (GB); Robert Peter Shellis, Avon (GB)

(21) Appl. No.: 12/744,881

(22) PCT Filed: Nov. 27, 2008
(86) PCT No.: PCT/EP2008/066314

§ 371 Date: Nov. 22, 2010

(30) Foreign Application Priority Data
Nov. 30, 2007 (GB) 0723523.7

(51) Int. Cl. A61K 8/69; C07K 14/00; C07K 14/77; C07K 14/415; A61K 8/64; A61Q 11/00

(52) U.S. Cl. 424/52; 530/360; 530/367; 530/350; 530/378; 424/49

(57) ABSTRACT

The present invention relates to the use of a protein selected from one or more of casein, ovalbumin, whey protein and soy protein for combating (i.e. helping to prevent, inhibit and/or treat) dental erosion and/or tooth wear caused by subsequent exposure to acid.

### NOVEL USE

The present invention relates to the use of a protein selected from one or more of casein, ovalbumin, whey protein and soy protein for combating (i.e. helping to prevent, inhibit and/or treat) dental erosion and/or tooth wear caused by subsequent exposure to acid.

Tooth mineral is composed predominantly of calcium hydroxyapatite, Ca₁₀(PO₄)₆(OH)₂, which may be partially substituted with anions such as carbonate or fluoride, and cations such as zinc or magnesium. Tooth mineral may also contain non-apatitic mineral phases such as octacalcium phosphate and calcium carbonate.

Tooth loss may occur as a result of dental caries, which is a multifactorial disease where bacterial acids such as lactic acid produce sub-surface demineralisation that does not fully remineralise, resulting in progressive tissue loss and eventually cavity formation. The presence of a plaque biofilm is a prerequisite for dental caries, and acidogenic bacteria such as Streptococcus mutans may become pathogenic when levels of easily fermentable carbohydrate, such as sucrose, are elevated in the oral cavity for extended periods of time.

Even in the absence of cariogenic disease, loss of dental hard tissue can occur as a result of acid erosion and/or physical tooth wear; these processes are believed to act synergistically. Exposure of the dental hard tissues to acid causes demineralisation, resulting in surface softening and a decrease in mineral density. Under normal physiological conditions, demineralised tissues self-repair through the remineralising effects of saliva. Saliva is supersaturated with respect to calcium and phosphate, and in healthy individuals saliva secretion serves to wash out the acid challenge, and raises the pH so as to alter the equilibrium in favour of mineral deposition.

Dental erosion (i.e. acid wear) is a surface phenomenon that involves demineralisation, and ultimately complete dissolution of the tooth surface by acids that are not of bacterial origin. Most commonly the acid will be of dietary origin, such as citric acid from fruit or carbonated drinks, phosphoric acid from cola drinks and acetic acid such as from vinegar. Dental erosion may also be caused by repeated contact with hydrochloric acid (HCl) produced by the stomach, which may enter the oral cavity through an involuntary response such as gastroesophageal reflux, or through an induced response as may be encountered in sufferers of bulimia.

Physical tooth wear is caused by attrition and/or abrasion. Attrition occurs when tooth surfaces rub against each other, a form of two-body wear. An often dramatic example is that observed in subjects with bruxism, a grinding habit where the applied forces are high, and is characterised by accelerated wear, particularly on the occlusal surfaces. Abrasion typically occurs as a result of three-body wear and the most common example is that associated with brushing with a toothpaste. In the case of fully mineralised enamel, levels of wear caused by commercially available toothpastes are minimal and of little or no clinical consequence. However, if enamel has been demineralised and softened by exposure to an erosive challenge, the enamel becomes more susceptible to tooth wear. Dentine is much softer than enamel and consequently is more susceptible to wear. Subjects with exposed dentine should avoid the use of highly abrasive toothpastes, such as those based on alumina. Again, softening of dentine by an erosive challenge will increase susceptibility of the tissue to wear.

Dentine is a vital tissue that in vivo is normally covered by enamel or cementum depending on the location i.e. crown versus root respectively. Dentine has a much higher organic content than enamel and its structure is characterised by the presence of fluid-filled tubules that run from the surface of the dentine-enamel or dentine-cementum junction to the odontoblast/pulp interface. It is widely accepted that the origins of dentine hypersensitivity relate to changes in fluid flow in exposed tubules, (the hydrodynamic theory), that result in stimulation of mechanoreceptors thought to be located close to the odontoblast/pulp interface. Not all exposed dentine is sensitive since it is generally covered with a smear layer; an occlusive mixture comprised predominantly of mineral and proteins derived from dentine itself, but also containing organic components from saliva. Over time, the lumen of the tubule may become progressively occluded with mineralised tissue. The formation of reparative dentine in response to trauma or chemical irritation of the pulp is also well documented. Nonetheless, an erosive challenge can remove the smear layer and tubule “plugs” causing outward dentinal fluid flow, making the dentine much more susceptible to external stimuli such as hot, cold and pressure. As previously indicated, an erosive challenge can also make the dentine surface much more susceptible to wear. In addition, dentine hypersensitivity worsens as the diameter of the exposed tubules increases, and since the tubule diameter increases as one proceeds in the direction of the odontoblast/pulp interface, progressive dentine wear can result in an increase in hypersensitivity, especially in cases where dentine wear is rapid.

Loss of the protective enamel layer through erosion and/or acid-mediated wear will expose the underlying dentine, and are therefore primary aetiological factors in the development of dentine hypersensitivity.

It has been claimed that an increased intake of dietary acids, and a move away from formalised meal times, has been accompanied by a rise in the incidence of dental erosion and tooth wear. In view of this, methods which help prevent dental erosion and tooth wear would be advantageous.

EP1 568 356A (Sara Lee), relates to the use of colostrum protein in oral care compositions for the prevention or reduction of dental erosion. The oral care compositions containing from 0.5 to 2.0 mg colostrum protein were shown to inhibit hydroxyapatite dissolution in 10 mM nitric acid, pH 5.0.

WO06/056013 (University of Melbourne), relates to a phosphopeptide-stabilised amorphous calcium phosphate and/or amorphous calcium fluoride phosphate complex useful in dental applications, including anti-erosion/corrosion, and in particular dental remineralisation.

The present invention is based on the discovery that certain proteins have the ability to combat (i.e. help to prevent, inhibit and/or treat) dental erosion caused by the intake of dietary acids.

The present invention provides the use of a protein selected from one or more of casein, ovalbumin, whey protein and soy protein in the manufacture of an oral care composition for combating dental erosion and/or tooth wear caused by subsequent exposure to acid i.e exposure to acid following administration of the protein(s).

In another aspect the present invention provides an oral care composition comprising a protein selected from one or more of casein, ovalbumin, whey protein and soy protein for use in combating dental erosion and/or tooth wear caused by subsequent exposure to acid. In one embodiment the protein is casein.

The protein for use in oral care compositions according to the invention is present in an amount of from 0.001% w/v to 10.0% w/v, suitably 0.01% w/v to 5.0% w/v, more suitably 0.10% w/v to 2.0% w/v.

Suitably the protein for use in the present invention is derived from natural sources.

Casein and whey protein are typically extracted from whole milk. Whey protein is a by-product of cheese manufacture. Ovalbumin is typically extracted from avian eggs, for example, chicken's eggs. Soy protein is typically extracted from soy beans.

An advantage presented by oral care compositions containing protein according to the invention, is that teeth when pre-treated with a composition according to the invention are protected against the damaging effect of subsequent exposure to dietary acid, typically in the pH range 2.0 to 4.5.

The term pre-treatment or pre-treated as used herein refers to treatment of the teeth with a composition comprising the protein prior to exposure to an acid.

Oral care compositions containing protein according to the invention will contain appropriate formulating agents such as abrasives, surfactants, thickening agents, humectants, flavouring agents, sweetening agents, opacifying or colouring agents, preservatives and water, selected from those conventionally used in the oral care composition art for such purposes. Examples of such agents are as described in EP 929287.

Preferably compositions containing protein for use according to the invention will further comprise a source of soluble fluoride ions such as those provided by sodium fluoride, sodium monofluorophosphate, tin (II) fluoride or an amine fluoride in an amount to provide from 25 ppm to 3500 ppm fluoride, preferably from 100 ppm to 1500 ppm.

Additional oral care actives may be included in the compositions containing protein according to the invention.

Oral care compositions containing protein for use according to the invention are typically formulated in the form of toothpastes, sprays, mouthwashes, gels, lozenges, chewing gums, tablets, pastilles, instant powders, oral strips or buccal patches.

The compositions containing protein for use according to the invention may be prepared by admixing the ingredients in the appropriate relative amount in any order that is convenient, and if necessary adjusting the pH to give a desired value for example from pH 5.5 to 9.0, suitably from pH 6.5 to 7.5.

In another aspect of the present invention there is provided a method of combating dental erosion and/or tooth wear caused by subsequent exposure to acid which comprises applying an effective amount of an oral care composition as hereinbefore defined to an individual in need thereof.

The present invention is illustrated by way of the following non-limiting examples.

### EXAMPLE 1

### To Demonstrate the Pre-Treatment Effect of a Composition Comprising Casein

### Materials and Method

### Hydroxyapatite Dissolution Measurements

An automatic titration system (Metrohm, Buckingham, UK), equipped with a 50 mL water-jacketed reaction vessel was set up to maintain constant pH by addition of 50 mmol/L HCl. The reaction temperature was 36° C. Hydroxyapatite was employed as an analogue of dental enamel (Barbour and Rees (2004) J. Dentistry 32: 591-602). Hydroxyapatite (HA) discs (Hitemco Medical Applications, Old Bethpage, USA), 12.05 mm in diameter×1.25 mm thick were fixed to a glass rod using sticky wax and the underside of the disc coated with nail varnish to give a constant exposed area of 155.5 mm² for each disc. The glass rod carrying the disc was fixed to a tube which fitted the inlet port of the reaction vessel lid and which held the specimen in a reproducible position with respect to the stirrer. For each dissolution measurement 15 mL of test solution was placed in the reaction vessel and stirred with a magnetic stirrer. When the temperature and pH had reached equilibrium the reaction was initiated by immersing the HA disc into the solution. Acid addition was linear with time and the rate of acid addition was used as the measure of HA dissolution.

New discs were conditioned by exposing them to stirred control citric acid solution for 30 min to remove any loosely attached or more soluble material. For every measurement, a control run and a test run were performed. The dissolution rate of an HA disc in the control citric acid solution was measured first and then the dissolution rate of the same HA disc in the test solution. Every measurement thus had its own control. Each test solution was tested three times. After a disc had been exposed to a protein test solution it was discarded.

To evaluate the persistence of the action of the protein against acid erosion, sequential measurements were performed. After conditioning the discs, three control runs of 30 minutes in the control citric acid solution were performed to obtain a mean baseline measurement. After this a single run of 2 minutes was performed with 0.10% w/v casein, pH 6.5. The discs were then washed and followed by sequential 30 minute runs in the control acid solution (pH 3.2) until the dissolution rate reached the baseline level. Each persistence of action experiment was performed at least three times with 0.10% w/v protein, each sequence being run on a separate HA disc.

Results

TABLE 1
Hydroxyapatite dissolution 0.3% w/v Citric acid, pH 3.2 over 240 min before
and after 0.10% w/v casein exposure. (Standard deviation in brackets).

| | | | | +30 | +60 | +90 | +120 | +150 | +180 | +210 | +240 | +270 |
|---|---|---|---|---|---|---|---|---|---|---|---|---|
| | control | control | control | min | min | min | min | min | min | min | min | min |
| 0.1% | 1.12 ± | 1.15 ± | 1.16 ± | 0.56 ± | 0.69 ± | 0.76 ± | 0.84 ± | 0.93 ± | 1.06 ± | 1.07 ± | 1.11 ± | 1.16 ± |
| sodium | 0.07 | 0.08 | 0.10 | 0.05 | 0.06 | 0.02 | 0.04 | 0.09 | 0.14 | 0.13 | 0.02 | 0.10 |
| caseinate | | | | | | | | | | | | |

### Summary

Example 1. demonstrates the pre-treatment effect where an inhibitory effect of casein against acid damage is apparent even after continuous immersion of the specimen in citric acid for 2.5 h.

### EXAMPLE 2

### Typical Dentifrice Formulation—2.0% w/v Protein

| Ingredient | Amount % w/v |
|---|---|
| Casein | 2.00 |
| Sorbitol | 30.00 |
| Thickening silica | 10.00 |
| Abrasive silica | 12.00 |
| Glycerin | 10.00 |
| Polyethylene glycol | 2.90 |
| Cocoamidopropyl betaine | 1.50 |
| Flavour | 1.00 |
| Sodium saccharin | 0.30 |
| Sodium fluoride | 0.24 |
| Titanium dioxide | 0.10 |
| Water | 29.96 |

1-3. (canceled)

4. An oral care composition comprising a protein selected from one or more of casein, ovalbumin, whey protein and soy protein combating dental erosion and/or tooth wear caused by subsequent exposure to acid.

5. An oral care composition according to claim 4 wherein the protein is present in an amount from 0.001% w/v to 10.0% w/v.

6. An oral care composition according to claim 4 further comprising a source of fluoride ions.

7. A method of combating dental erosion and/or tooth wear caused by subsequent exposure to acid which comprises applying an effective amount of an oral care composition as defined in claim 1 to an individual in need thereof.
